(19) United States
(12) Patent Application Publication
(10) Pub. No.: US 2008/0027556 A1
(43) Pub. Date: Jan. 31, 2008

(54) Compliant tibial component

(75) Inventor: Robert Metzger, Wakerusa, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 11/483,699

(22) Filed: Jul. 10, 2006

(51) Int. Cl. A61F 2/38; B29C 41/20; B22F 3/11; B22F 3/24; B22F 7/00

(52) U.S. Cl. 623/20.32; 264/259; 419/2; 419/9; 419/10; 419/28

(57) ABSTRACT

A tibial component. The tibial component includes a discontinuous metal backing formed by a plurality of discrete metal elements, and a non-metal bearing secured to the discontinuous metal backing.

### INTRODUCTION

Several tibial components are available for use in knee arthroplasty for use with bone cement or in cementless applications. Such components include all-metallic, all-polyethylene or metallic with polyethylene bearing components.

Although the existing tibial components can be satisfactory for their intended purposes, there is still a need for improved tibial components.

### SUMMARY

The present teachings provide a tibial component. The tibial component includes a discontinuous metal backing formed by a plurality of discrete metal elements, and a non-metal bearing secured to the discontinuous metal backing.

The present teachings provide a tibial component including a discontinuous metal backing, and a non-metal bearing secured to the metal backing, the metal backing imparting compliance to the tibial component as compared to a continuous metal backing.

The present teachings provide a method of making a tibial component. The method includes sintering porous metal powder into solid metal to form a metal structure having a porous metal base and a solid metal layer, machining the metal structure into discrete metal elements, and molding a non-metal tibial bearing onto the solid metal layers of the discrete metal elements.

Further areas of applicability of the present invention will become apparent from the description provided hereinafter. It should be understood that the description and specific examples are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective bottom view of a tibial component according to the present teachings;

FIG. 2 is a perspective bottom view of a tibial component according to the present teachings;

FIG. 3 is a side view of a tibial component according to the present teachings;

FIG. 4 is a side view of a tibial component according to the present teachings;

FIG. 5 is a perspective view of one-half of a mold illustrating a discrete metal backing element of a tibial component according to the present teachings;

FIG. 6 is a perspective view of a mold illustrating a discrete metal backing element of a tibial component according to the present teachings; and

FIG. 7 is a sectional view of a mold illustrating a discrete metal backing element of a tibial component according to the present teachings.

### DESCRIPTION OF VARIOUS ASPECTS

The following description is merely exemplary in nature and is in no way intended to limit the invention, its application, or uses. For example, although the present teachings are illustrated for applications cruciate retaining applications in knee surgery, the present teachings can be used for partial or total knee replacements both in primary and revision instances with cruciate retaining, posterior stabilized, or posterior stabilized with additional constraint options, for example.

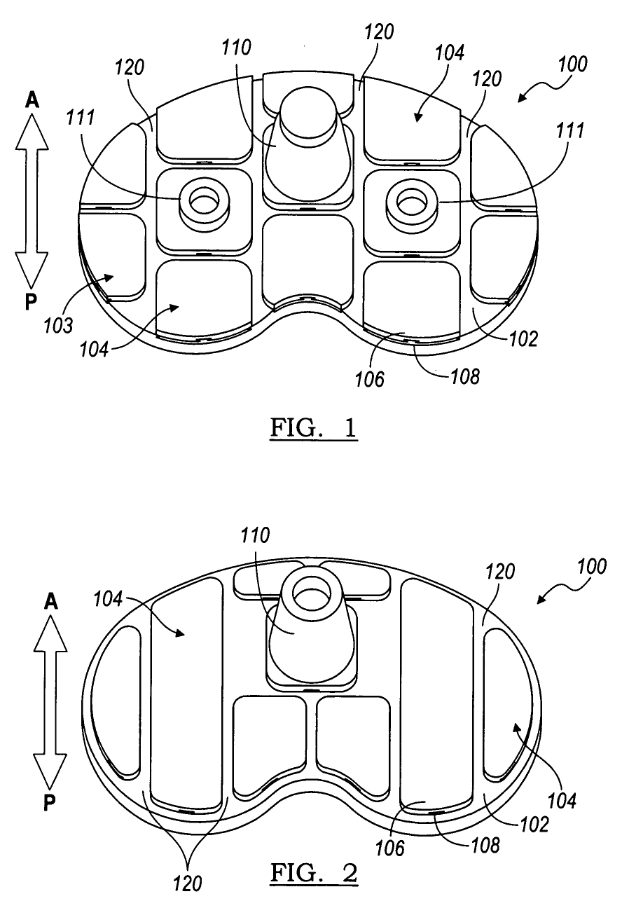

Referring to FIG. 1, an exemplary tibial component 100 according to the present teachings includes a non-metal bearing 102 molded onto a metal backing 103. The metal backing 103 is discontinuous, being defined by a plurality of discrete (not interconnected) metal elements 104. The discrete metal elements 104 can be substantially parallel and oriented in columns along the antero-posterior direction AP. Other orientations and patterns can also be used for the metal elements 104. The discrete metal elements 104 can be elongated along the antero-posterior direction in the form of strips, as shown in FIG. 2, or can have be blocks having aspect ratio of about 1, such as short or square-like blocks, as shown in FIG. 1.

Referring to FIGS. 1 and 2, the discrete elements 104, whether in the form of strips or blocks, can be aligned in columns along the antero-posterior direction, such that the discrete elements 104 are separated by AP-oriented elongated bearing strips 120 of exposed non-metal bearing. The lack of metal backing 103 along the bearing strips 120 can increase the compliance of the tibial component 100 in the anterior--posterior direction AP and in the medial-lateral direction, as compared to an all metal backing or to a continuously-interconnected backing. Compliance, as used herein, includes the ability to flex or bend and otherwise deform elastically or non-permanently in various directions, enabling the component to better conform to the bone shape and or follow bone movements.

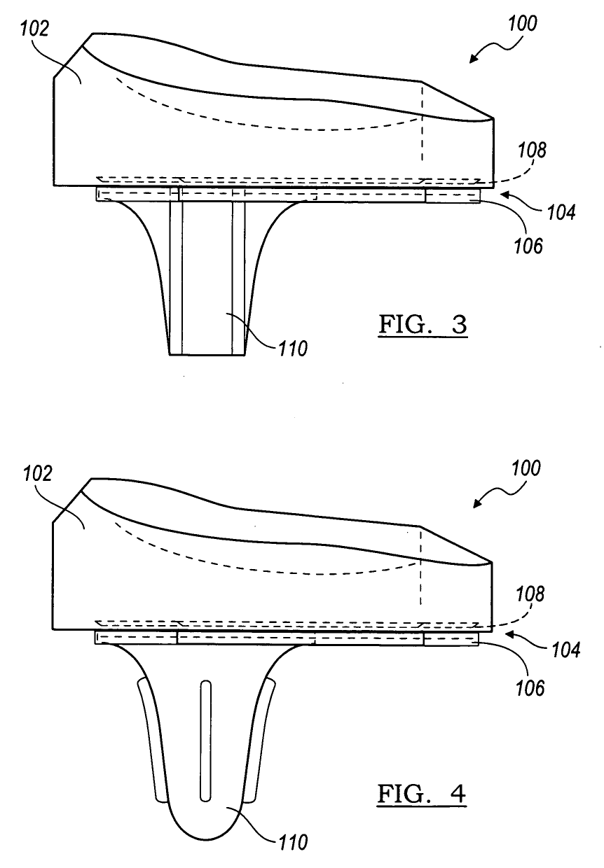

Referring to FIGS. 1-4, the tibial component can include bone fixation elements, such as a stem 110 and optional posts or pegs 111. The stem 110 can be made as one integral piece or from modular components. The stem 110 can be tapered. The stem 110 and the posts 111 can be metal or non-metal. The non-metal bearing 102 can be polymer or other biocompatible plastic.

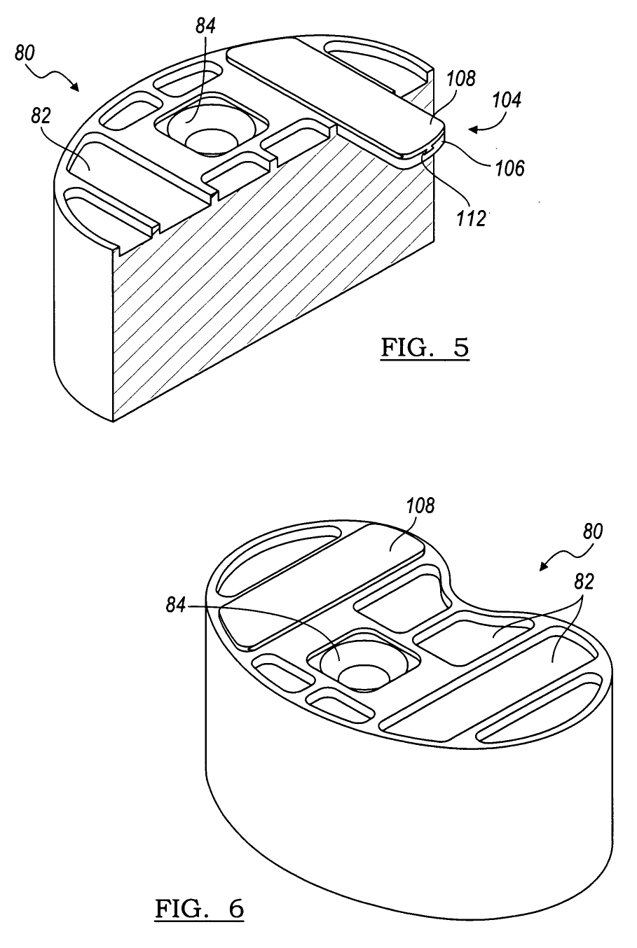

Each discrete element 104 of the metal backing 103 can include a porous metal base 106 overlaid with a solid metal layer 108. The base 106 and the layer 108 can be mechanically interconnected, such as with a dovetail interconnection or other interlocking connections 112, as shown in FIG. 5. The porous metal of the base 106 and the solid metal of the layer 108 can be titanium, titanium alloys, such as Ti-6AI-4V, or other biocompatible metals or alloys. The porous metal base 106 can allow for cementless fixation of the tibial component 100.

The discrete elements 104 of the metal backing 103 can be made by pressing porous metal/metal powder base 106 to the solid metal layer 108, sintering the entire structure to bond the materials, and machining the resulting backing 103 into strips or blocks 104 of desired size and shape. Before sintering, the solid metal layer 108 can be grit-blasted on the surface that mates with the porous metal base 106 to create a rough surface promoting bond. The discrete metal elements 104 can also be made by sintering metal powder together such that a gradient from porous to solid is created. The discrete metal element 104 can also be made by taking a block of porous metal and smearing the top layer to close the pores and make the top layer solid.

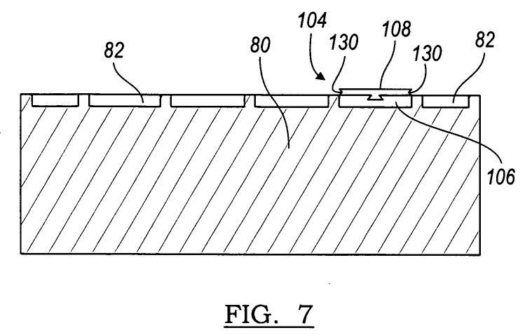

Referring to FIGS. 5-7, aspects of the molding process for making the tibial component 100 are illustrated. A mold 80 that includes recesses 82 for receiving the discrete elements 104 and an opening 84 for receiving the stem 110 can be provided. After the discrete elements 104 are fixed in the recesses 82 of the mold 80, polymer or other plastic material is molded over the solid metal layer 108 without interfacing with the porous metal base 106 to form the non-metal bearing 102. The solid metal layer 108 can include grooves or other interlocking formations 130 for interfacing with the non-metal bearing 102.

It will be appreciated that the discrete metal elements 104 can increase the compliance of the tibial component in the anterior-posterior and medial lateral direction and can allow the tibial component to flex. Further, the discrete metal elements 104 and their porous metal bases 106 can promote tissue ingrowth. The porous metal bases 106 can allow cementless fixation.

The foregoing discussion discloses and describes merely exemplary arrangements of the present invention. One skilled in the art will readily recognize from such discussion, and from the accompanying drawings and claims, that various changes, modifications and variations can be made therein without departing from the spirit and scope of the invention as defined in the following claims.

What is claimed is:

1. A tibial component comprising:
a discontinuous metal backing formed by a plurality of discrete metal elements; and
a non-metal bearing secured to the discontinuous metal backing,

2. The tibial component of claim 1, wherein each discrete metal element includes a porous metal base and a solid metal layer, the solid metal layer having a first surface coupled to the porous metal base and a second surface coupled exclusively to the non-metal bearing.

3. The tibial component of claim 2, wherein the porous metal base and a solid metal layer are mechanically coupled to one another with an interlock connection.

4. The tibial component of claim 2, wherein the interlock connection is a dovetail connection.

5. The tibial component of claim 3, wherein the porous metal base and the solid metal layer are sintered to one another.

6. The tibial component of claim 1, wherein the discrete elements are elongated strips.

7. The tibial component of claim 6, wherein the elongated strips are arranged substantially parallel to an anterior-posterior direction of the tibial component.

8. The tibial component of claim 1, wherein the discrete elements are blocks of aspect ratio substantially equal to about one.

9. The tibial component of claim 8, wherein the blocks are arranged in columns, the columns substantially oriented along an anterior-posterior direction of the tibial component.

10. The tibial component of claim 1, further including a fixation stem.

11. The tibial component of claim 10, further including a fixation post.

12. The tibial component of claim 1, further including a taper for a modular stem.

13. The tibial component of claim 1, wherein the non-metal component is polymeric.

14. A tibial component comprising:
a discontinuous metal backing; and
a non-metal bearing secured to the metal backing, the discontinuous metal backing increasing the compliance of the tibial component as compared to a continuous metal backing.

15. The tibial component of claim 14, wherein the metal backing is formed by a plurality of discrete metal elements.

16. The tibial component of claim 15, wherein each discrete metal element includes a porous metal base and a solid metal layer, the solid metal layer having a first surface coupled to the porous metal base and a second surface coupled exclusively to the non-metal bearing.

17. The tibial component of claim 14, wherein the non-metal bearing is molded to the metal backing.

18. A method of making a tibial component, the method comprising:
sintering porous metal powder into solid metal to form a metal structure having a porous metal base and a solid metal layer;
machining the metal structure into discrete metal elements; and
molding a non-metal tibial bearing onto the solid metal layers of the discrete metal elements.

19. The method of claim 18, further comprising aligning the discrete metal elements in an anterior-posterior direction relative to the tibial component.

20. The method of claim 18, further comprising arranging the discrete metal elements in column along an anterior posterior direction of the tibial component.

21. The method of claim 18, further comprising roughening a surface of the solid metal layer such that the roughened surface contacts the porous metal.

22. The method of claim 18, wherein molding excludes interfacing the non-metal bearing with the porous metal base.
